(19) United States
(12) Patent Application Publication
(10) Pub. No.: US 2025/0279196 A1
(43) Pub. Date: Sep. 4, 2025

(54) MEDICAL DEVICE, SYSTEM AND METHOD FOR ALARM MANAGEMENT

(71) Applicant: Fresenius Vial SAS, Brézins (FR)

(72) Inventor: Didier Bosse-Platiere, Les Abrets (FR)

(21) Appl. No.: 18/863,741

(22) PCT Filed: Jul. 27, 2023
(86) PCT No.: PCT/EP2023/070808

§ 371 Date: Nov. 7, 2024

(30) Foreign Application Priority Data
Jul. 29, 2022 (EP) 22315176.2

(51) Int. Cl. G16H 40/63; A61M 5/142

(52) U.S. Cl. CPC G16H 40/63; A61M 5/142; A61M 2205/18; A61M 2205/3561; A61M 2205/3592; A61M 2205/50; A61M 2205/581; A61M 2205/583

(57) ABSTRACT

A medical device (1A-1E, 2) comprises a communication interface (10, 20) communicatively couplable or coupled with another device (2, 1A-1E), and a controller (11, 21) configured to be selectively operated in one of a plurality of predetermined alarm modes, wherein the controller (11, 21) is further configured to: determine in which operational setup (A-E) of a plurality of predetermined operational setups (A-E) the medical device (1A-1E, 2) is operating; determine a quality of service of a communication with the other device (2, 1A-1E) via the communication interface (10, 20); select one of the plurality of predetermined alarm modes based on the determined operational setup (A-E) and the determined quality of service; and operate the medical device (1A-1E, 2) in the selected alarm mode.

The invention relates to a medical device, to a system and to a method for alarm management.

In the health sector in general, and specifically in hospitals, patients and various medical devices are continuously monitored. In many cases, e.g., when a monitored parameter of a patient exceeds a certain threshold or when a medical device malfunction is detected, alarms are produced to alert a caregiver. The caregiver then attends the alarm, performs a corresponding action and stops the alarm. To ensure that a relevant alarm is not overlooked, many alarms are audible alarms which are hearable, e.g., in a patient room at a bedside of the patient. Nowadays, commonly there is a significant noise pollution by numerous alarms, particularly at intensive care units. Such noise pollution can increase stress levels of patients and caregivers.

R. R. Vitoux et al. describe this noise pollution in “Perceptions of Infusion Pump Alarms”, J Infus Nurs. 2018 September; 41(5): 309-318, published Sep. 14, 2018 and propose potential solutions including identifying non-decibel noise-producing alternatives to alert nurses to a patient's alarming pump; employing real-time dashboards, alarm prioritization, patient-specific alarm customization technologies, and educating nurses on specific practices and pump configurations to help reduce unnecessary alarms.

WO 2021/207280 A1 describes that in many cases, depending upon the type of therapy being delivered (including the type of drug being delivered by an infusion pump) it may not be necessary to activate a particular alarm when an error condition is detected.

Such solutions can contribute to reduce noise pollution in the use of medical devices, but the effects are limited to certain types of alarms and clinical situations.

WO 2017/173162 A1 proposes to reduce alert fatigue by providing a user-selected mute button and applying an escalating mute override feature that can override the user-selected mute button or do not disturb mode for alarms in an escalating manner as follows. When a high alert is triggered, an app generates an alarm to cause a vibrate action produced by a smartphone. If the user does not acknowledge the alert in a predetermined time (e.g., 5 minutes), then the app generates a second alarm as a low-volume audio alarm produced by the smartphone. If the user does not acknowledge the high alert associated with the second alarm in a second predetermined time (e.g., another 5 minutes), then the app generates a third alarm as a high-volume audio alarm produced by the smartphone. This solution is limited to certain situations and can increase delays.

It is an object of the instant invention to further reduce noise pollution in the context of the application of medical devices.

This object is achieved by means of a medical device comprising the features of claim 1.

Accordingly, a (first) medical device is provided, comprising a communication interface and a controller. The communication interface is communicatively couplable or coupled with another (i.e., second) device, e.g., a point of care medical device. The controller is configured to be selectively operated in one of a plurality of predetermined alarm modes. The controller is further configured to: determine in which operational setup of a plurality of predetermined operational setups the (first) medical device is operating; determine a quality of service of a communication with the other device (e.g., the point of care medical device) via the communication interface; select one of the plurality of predetermined alarm modes based on the determined operational setup and the determined quality of service; and operate the (first) medical device in the selected alarm mode.

This is based on various findings. Firstly, it has been found that for every patient and corresponding operational setup the requirements for an alarm reporting may change, so that a proper alarm configuration can be time consuming for caregivers. Secondly, it has been recognized that certain operational setups of the (first) medical device, e.g., an intensive-care setup, require stricter alarm reporting and acknowledgement than other operational setups, and, thirdly, that a limiting factor of an automatic reduction of alarms, e.g., at the bedside, is the reliability of the communication between the (first) medical device and the other (second) device. By determining the quality of service of the communication with the other (second) device, a measure of the reliability of a remote alarm production is acquired. If a high quality of service is determined, for example, alarms may be muted at the (first) medical device and instead only produced at the other (second) device. As another example, if an intermediate quality of service is determined, alarms may be muted at the (first) medical device only in an operational setup with low criticality, but may be produced, e.g., in an intensive-care operational setup. Thus, the combination of the automatic detection of the current operational setup and the determination of the communication quality of service allows an automatic alarm mode selection. This enables a significant reduction of noise pollution in the context of the application of medical devices, and additionally allows to reduce the workload of caregivers by reduced configuration requirements.

To give an example, the plurality of predetermined operational setups may comprise an intensive-care setup, an operating room setup and a ward level setup. As another example, the plurality of predetermined operational setups may be defined to comprise a high criticality care setup (requiring high caregiver attention) and a low criticality care setup (requiring little caregiver attention), and, optionally, an intermediate criticality care setup (requiring an intermediate level of caregiver attention).

The (first) medical device may be an infusion device or an infusion station for administering one or more fluids to a patient. Particularly for infusion devices and stations the described alarm management allows a reduction of the noise with a high level of safety.

The controller may be further configured to delegate or disseminate an alarm message to the other (second) device, e.g., depending on the determined operational setup and/or the determined quality of service. Thus, the corresponding alarm may be muted at the (first) medical device itself. Since the (first) medical device may be arranged at the bedside, this allows to reduce the noise for the patient while maintaining a high security level. Notably, the alarm message handling based on the quality of service particularly allows a noise reduction at a high level of security.

Optionally, the controller is further configured, in case of a delegated message, to receive a response to the communicated alarm message from the other (second) device. The production of an alarm by the (first) medical device may depend on the reception and/or content of the response. This allows to assure an uninterrupted communication.

The controller may be further configured to produce a visible alarm and/or an audible alarm based on a delay of a response to the communicated alarm message and/or based on the selected alarm mode. This allows to assure a fast response of a caregiver irrespective of the communication quality of service.

The (first) medical device optionally comprise a display adapted to produce a visible alarm.

This allows to visibly display alarms instead of audible alarms to reduce the noise level, and to additionally produce alarms to increase the level of attention.

The (first) medical device may comprise a sound emitting device, such as a loudspeaker or beeper, adapted to produce an audible alarm. Thus, the (first) medical device itself may produce the alarm, if necessary.

For example, the plurality of predetermined alarm modes comprise a suppression mode, in which alarms are suppressed, and a perception mode, in which the alarms are perceivable. For example, for a good quality of service and in a ward level setup, the suppression mode may be selected, so that alarms at the (first) medical device itself are suppressed.

Optionally, the plurality of predetermined alarm modes further comprises an attenuation mode, in which alarms are attenuated. For example, for an intermediate quality of service and in a ward level setup, the attenuation mode may be selected, so that alarms at the (first) medical device itself are attenuated.

The controller may be configured to determine in which operational setup of the plurality of predetermined operational setups the (first) medical device is operating by at least one of: receiving a user input at the (first) medical device; receiving a message indicating an operational setup via the communication interface; and detecting a predetermined device or a predetermined plurality of devices via the communication interface. This allows a simple (or even fully automatic) configuration of the (first) medical device.

The controller may be further configured to determine the quality of service of a communication with the other device (second device) by at least one of: receiving a response from the other (second) device; determining that a latency of a communication with the other (second) device is above or below a predetermined latency threshold; and determining that a network bandwidth of a communication with the other (second) device is above or below a predetermined network bandwidth threshold. This allows to automatically determine the quality of service in order to select the respective alarm mode. Optionally, the controller is configured to determine the quality of service and, if necessary, change the alarm mode accordingly, on a regular basis. For example, the controller is configured to repeat these steps periodically, with a time interval that may be, e.g., predefined or determined based on the quality of service. This allows to ensure that always the appropriate alarm mode is set.

The predetermined operational setups may comprise at least one wireless setup, in which the communication interface communicates via a wireless connection with the other (second) device, and at least one wired setup, in which the communication interface communicates via a wired connection with the other (second) device. This allows a wide range of applications.

According to an aspect, a system is provided, the system comprising at least one (first) medical device in accordance with any embodiment described herein and the other (second) device communicatively coupled with the at least one (first) medical device. For the advantages, reference is made to the above description of the (first) medical device.

The system may comprise a plurality of (first) medical devices communicatively coupled with the other (second) device via a same network. Thus, the other (second) device may acquire alarms from the plurality of (first) medical devices and, e.g., structure the alarms in a quickly comprehensible manner.

The other (second) device may be a caregiver device adapted to visibly display and/or audibly emit alarms, and/or to send alarms to a mobile device. This allows a quick response by the caregiver. The other (second) device may be a patient monitor, a nursing station, a ventilator, a dedicated PC software application or the like.

According to an aspect, a method for operating a (first) medical device is provided. The (first) medical device comprises a communication interface communicatively couplable or coupled with another (a second) device. The (first) medical device further comprises a controller configured to be selectively operated in one of a plurality of predetermined alarm modes, the method comprising: determining (e.g., by the (first) medical device) in which operational setup of a plurality of predetermined operational setups the (first) medical device is operating; determining (e.g., by the (first) medical device) a quality of service of a communication with the other (second) device via the communication interface; selecting (e.g., by the (first) medical device) one of the plurality of predetermined alarm modes based on the determined operational setup and the determined quality of service; and operating the (first) medical device in the selected alarm mode.

The method may use the (first) medical device and/or the system in accordance with any embodiment described herein. Regarding the advantages of the method reference is made to the description of the (first) medical device and system above.

The idea underlying the invention shall subsequently be described in more detail by referring to the embodiments shown in the figures. Herein:

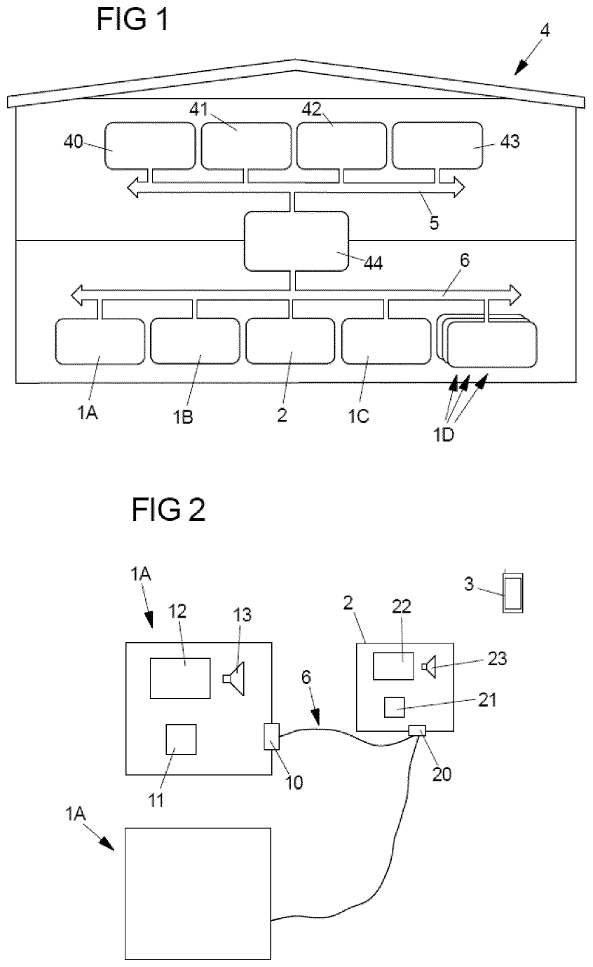

FIG. 1 shows a system for alarm management with various medical devices;

FIG. 2 shows first medical devices, a second device and a mobile device of the system of FIG. 1;

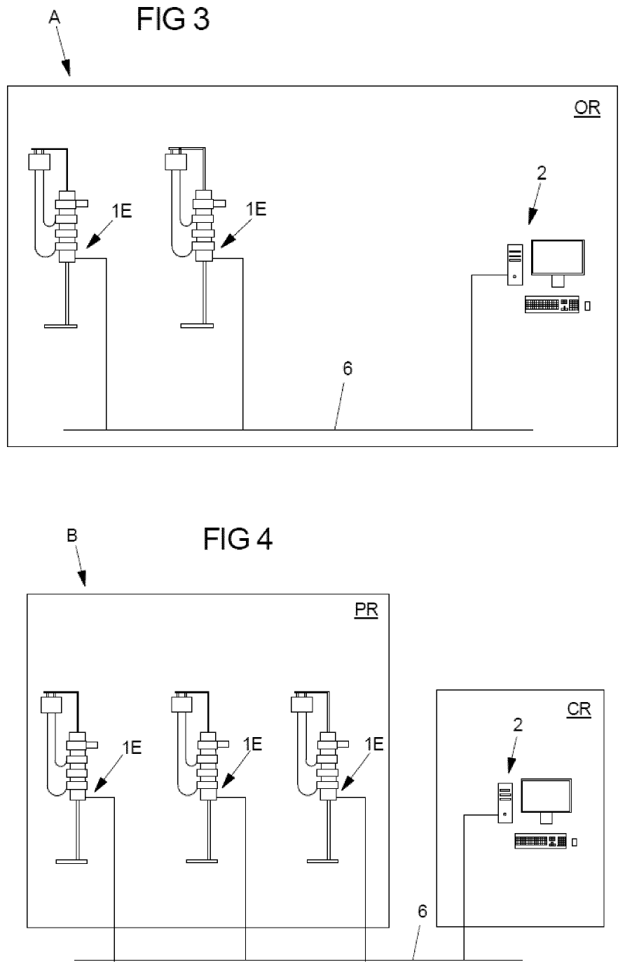

FIG. 3-7 show different operational setups of several first medical devices and a second device of the system of FIG. 1; and

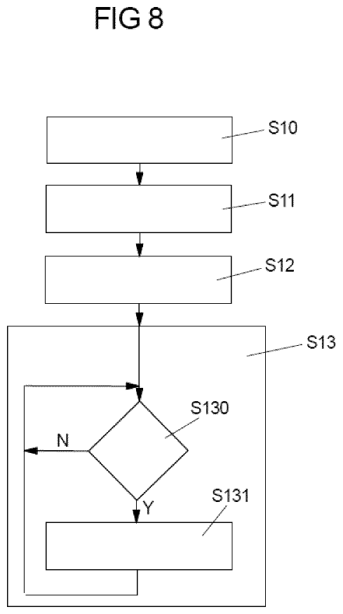

FIG. 8 shows a method for operating one of the medical devices of FIG. 1.

Subsequently, a medical device, a system and a method for operating a medical device, particularly for alarm management shall be described in certain embodiments. The embodiments described herein shall not be construed as limiting for the scope of the invention.

FIG. 1 shows a system 4 for alarm management of a hospital. The system 4 comprises various medical devices, in the following referred to as first medical devices 1A-1D, and another device, in the following referred to as second device 2 (optionally a second medical device), communicatively coupled with one another via a monitoring network 6. The first medical devices 1A-1D and/or the second device 2 may be arranged at the bedside of a patient. The second device 2 may be a point of care medical device.

Further, the system 4 comprises a hospital information system 40, HIS. The hospital information system 40 is an information system designed to manage medical, administrative, financial, and/or legal issues and corresponding processing of services of the hospital's operation. The system 4 further comprises a picture archiving and communication system 41, PACS. The picture archiving and communication system 41 provides storage and access to images. The system 4 further comprises a clinical information system 42, CIS. The clinical information system 42 is an information system for use in a critical care environment, such as in an intensive care unit, ICU. It can network with different devices of the system 4, and draw information from these devices into an electronic patient record, which clinicians can see at the patient's bedside. Further, the system 4 comprises a laboratory device 43, e.g., a computer system storing and/or processing laboratory data. The hospital information system 40, the picture archiving and communication system 41, the clinical information system 42 and the laboratory device 43 are communicatively coupled with one another via a hospital network 5.

The hospital network 5 and the monitoring network 6 are communicatively coupled with one another via a communication device 44, which may be a central station, a gateway and/or a bedside device data aggregator.

First medical device 1A is an infusion pump (alternatively, an infusion station) for administering at least one fluid to a patient. First medical device 1B is a dialysis machine. First medical device 1C is an anaesthesia workstation. First medical devices 1D are surgical devices. In general, the system 4 comprises at least one first medical device 1A-1D and at least one second device 2. It may further comprise one or more, e.g., all of the other devices illustrated in FIG. 1.

With reference to FIG. 2, the functioning of one of the first medical devices 1A-1D, first medical device 1A, and of the system 4 will now be described in more detail.

As shown in FIG. 2, first medical device 1A comprises a communication interface 10 communicatively couplable or coupled with the second device 2. The first medical device 1A further comprises a controller 11 configured to be selectively operated in one of a plurality of predetermined alarm modes, an optional display 12, and a sound emitting device 13. In the present example, the sound emitting device 13 is a loudspeaker. The controller 11 is operatively coupled with the display 12 and the sound emitting device 13. The controller 11 is adapted to control the display 12 and/or the sound emitting device 13 to produce an alarm (visible and/or audible).

In the present example, the second device 2, which in this example is a bedside patient monitor, comprises a communication interface 20 communicatively couplable or coupled with the first medical device 1A. The second device 2 further comprises a controller 21 configured to be selectively operated in one of a plurality of predetermined alarm modes, an optional display 22, and a sound emitting device 23. In the present example, the sound emitting device 23 is a loudspeaker. The controller 21 is operatively coupled with the display 22 and the sound emitting device 23. The controller 21 is adapted to control the display 22 and/or the sound emitting device 23 to produce an alarm (visible and/or audible).

The first medical device 1A and the second device 2 are communicatively coupled with one another by means of their respective communication interfaces 10, 20 and via the monitoring network 6.

The controller 11 of the first medical device 1A (optionally, also the controller 21 of the second device 2) is further configured to determine in which operational setup of a plurality of predetermined operational setups the first medical device 1A is currently operating. Various different operating setups A-E will be described in more detail below with reference to FIGS. 3-7. In general, the predefined operational setups may comprise setups with different levels of requirements (e.g., low and high, or low, intermediate and high), e.g., setups with a different criticality of care, with a different flow of information and/or with different setups of communicative connections, for example.

The controller 11 of the first medical device 1A (optionally, also the controller 21 of the second device 2 in a corresponding manner) is further configured to determine a quality of service, QoS, of a communication with the second device 2 via the communication interface 10. The controller 11 of the first medical device 1A may be adapted to perform one or more quality measurements. For example, the controller 11 may be adapted to measure a latency, a bandwidth, a signal strength, a bit error rate or the like to determine the QoS. Optionally, the QoS may be determined to be in one of a plurality of different QoS levels (e.g., low and high, or low, intermediate and high).

The controller 11 of the first medical device 1A (optionally, also the controller 21 of the second device 2) is further configured to select one of the plurality of predetermined alarm modes based on the determined operational setup and the determined quality of service. For example, the controller 11 may access a (e.g., predefined) look-up table, LUT, which outputs one of the plurality of predetermined alarm modes upon input of the determined operational setup and the determined quality of service.

The controller 11 of the first medical device 1A (optionally, also the controller 21 of the second device 2) is further configured to operate the first medical device 1A (or second device 2) in the selected alarm mode. Two, three or more alarm modes may be provided, which differ from one another by the amount, length, sound frequency, repetition frequency, volume and/or luminosity of alarms produced in response to the same type of alarm message. For example, the plurality of predetermined alarm modes comprises a suppression mode, in which alarms are suppressed, an attenuation mode, in which alarms are perceivable attenuated, and a perception mode, in which the alarms are perceivable (not attenuated). As another example, the alarm modes may comprise a local alarm only mode and a remote alarm only mode. In case of a QoS getting worse, the alarm mode may dynamically be changed.

For example, upon setting up the system 4, the controller 11 of the first medical device 1A determines in which operational setup of the plurality of predetermined operational setups the first medical device 1A is operating. This may be performed, e.g., by receiving a user input at the first medical device 1A or the second device 2, by receiving, by the controller 11, a message indicating an operational setup via the communication interface 10, for example from the second device 2, and/or by detecting a predetermined device or a predetermined plurality of devices, e.g., via the communication interface 10.

The controller 11 of the first medical device 1A then determines the QoS of the communication with the second device 2, e.g., by receiving, or not receiving, a response from the second device 2 to a query (e.g., within a predetermined period of time), by determining that a latency of a communication with the second device 2 is above or below a predetermined latency threshold, and by determining that a network bandwidth of a communication with the second device 2 is above or below a predetermined network bandwidth threshold. The controller 11 may determine the QoS by determining whether the communicative connection with the second device 2 is functioning, i.e., whether the second device 2 is reachable. Optionally, the controller 11 of the first medical device 1A then categorizes the QoS in one of a set of predefined categories (e.g., low, average, high).

The controller 11 of the first medical device 1A then selects one of the plurality of predetermined alarm modes based on the determined operational setup and the determined QoS, e.g., based on a given rule that assigns alarm modes to the operational setup and QoS. Then, the first medical device 1A operates in the selected alarm mode and produces alarms as defined in the selected alarm mode. Optionally, the selected alarm mode is displayed at the first medical device 1A and/or the second device 2.

When the controller 11 of the first medical device 1A operates in the selected alarm mode and processes (e.g., receives or generates) an alarm message, an alarm is produced by means of the display 12 and/or the sound emitting device 13 based on the alarm message and the selected alarm mode. For example, an alarm indicated in the alarm message may only be produced when the selected alarm mode is the perception mode and suppressed when the selected alarm mode is the suppression mode. Thus, if the QoS is good, and depending on the operational setup, some or all of the alarm messages at the controller 11 may be delegated or disseminated by the controller 11 to the second device 2. The second device 2 then produces an alarm in accordance with the alarm message and/or forwards the alarm message (or sends a corresponding message, e.g., SMS) to mobile device 3. However, the first medical device 1A may require the reception of a response to a delegated alarm message. If the response is delayed, e.g., by more than a predefined time period and depending on the selected alarm mode, the controller 11 of the first medical device 1A produces a visible and/or audible alarm.

Optionally, the alarm mode can be changed remotely, e.g., via the second device 2. The first medical device 1A, second device 2 and system 4 can be compliant with alarm standard IEC/EN 606601 Jan. 8.

Optionally, the controller 11 of the first medical device 1A determines the QoS of the communication with the second device 2 periodically. For example, the controller 11 of the first medical device 1A determines the QoS of the communication with the second device 2 once per minute. The alarm mode may be dynamically adjusted in accordance with the periodically determined QoS.

In accordance with FIG. 2, the system comprises a further first medical device 1A which is designed as the first medical device 1A described above.

FIG. 3 shows a first exemplary operational setup A. The operational setup A is that of an operating room OR. Various first medical devices 1E, here each in the form of a centralized management device managing several infusion pumps, are communicatively coupled with a second device 2, e.g., a monitoring device, via the monitoring network 6. Here the monitoring network 6 is a wired network with a low latency, high bandwidth and, thus, a high QoS.

The selected alarm mode takes the high QoS and the need for a quiet operating room into account. The controllers of the first medical devices 1E are configured in the selected alarm mode with limited noise and luminosity. The QoS is monitored regularly. A Service-oriented Device Connectivity (SDC), e.g., in accordance with IEEE 11073, is used for bidirectional safe and timeout-limited information exchange. This allows near real-time bidirectional communication. The selected alarm mode complies with a Distributed Alarm System (DAS), where a service is guaranteed, or with a Confirmation of a Distributed Alarm System (CDAS), where a confirmation of a received alarm is provided.

FIG. 4 shows another exemplary operational setup B. The operational setup B is that of a wired connection at ward level. Several first medical devices 1E are arranged in a patient room PR, while a second device 2 is arranged in a caregiver room CR. Here, a quiet patient room PR is wanted and infusion information including alarms are reported by the first medical devices 1E to the second device 2. The second device 2 produces the alarms visually and/or audibly. The QoS is good due to the wired network connection. Therefore, the first medical devices 1E select an alarm mode according to which the first medical devices 1E do not produce the alarms. A Distributed Information System (DIS) is used as alarm reporting system. Hence, in connection with the alarm mode selection, an alarm reporting system may be selected based on the determined operational setup and the determined QoS, e.g., among a set of predetermined alarm reporting systems, such as DIS, DAS and CDAS.

A proprietary communications protocol may be applied, e.g., through an OpenVPN server.

FIG. 5 shows another exemplary operational setup C. The operational setup C is that of a wireless virtual switching system in a private network, but with low criticality. Several first medical devices 1A at the patient's bedside are connected wirelessly to the monitoring network 6. A second device 2 in the form of a standard PC with monitor. Due to the wireless connection, the QoS may be low. However, since also the criticality of the operational setup C is low, an alarm mode with no or attenuated alarm levels at the first medical devices 1A and alarm forwarding to the second device 2 with a DIS alarm reporting system are selected. HTTPS may be used as protocol for the alarm reporting. The communications may be controlled by a server 7.

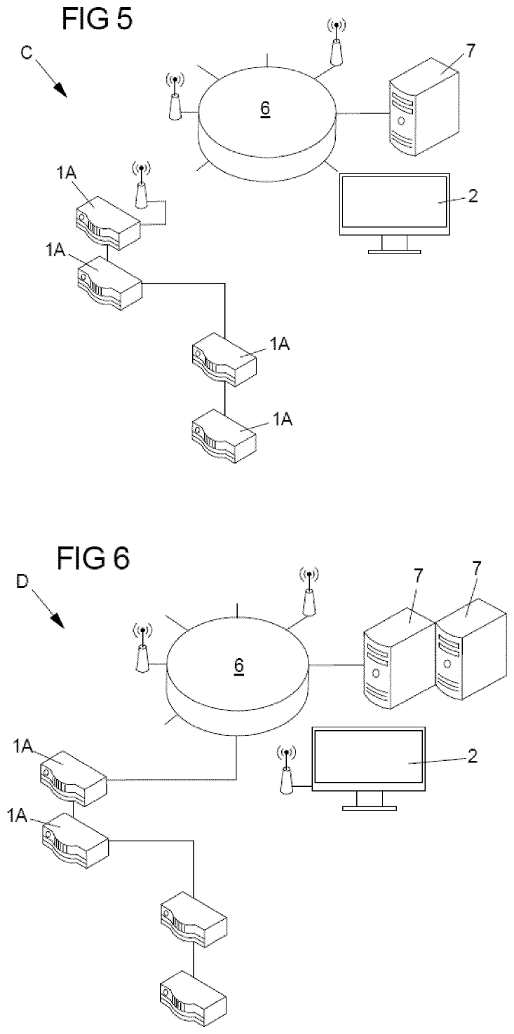

FIG. 6 shows another exemplary operational setup D. The operational setup D is that of a wireless setup with an HL7 network. For the HL7 protocol, profile HL7 ACM may be used. In this example, the second device 2 is wirelessly connected to the monitoring network 6. One or more servers 7 may provide a DAS or a CDAS functionality. Alternatively, DIS is used, depending on the QoS. The criticality of the operational setup is low.

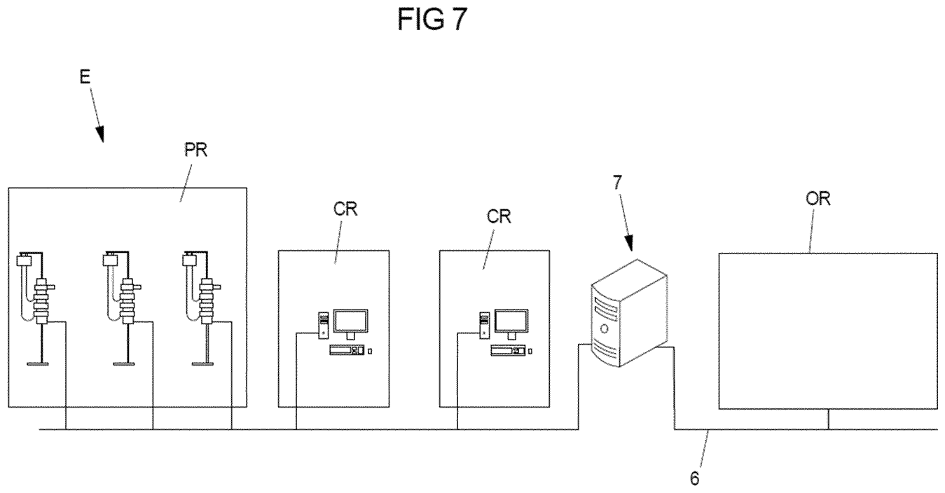

FIG. 7 shows another exemplary operational setup E. The operational setup E is that of two bridged operational setups. Therein a patient room PR and caregiver rooms CR with first medical devices and second devices are illustrated which are connected via a network to a bridging server 7. Further, an operating room OR is connected via another monitoring network 6 to the server 7. Alarms may be exchanged between the two parts, but a response lag is expected. The first medical devices will therefore select an alarm mode that produces perceivable alarms.

FIG. 8 shows a method for operating a first medical device, the first medical device comprising a communication interface communicatively couplable or coupled with a second device, and a controller configured to be selectively operated in one of a plurality of predetermined alarm modes. The method comprises the following steps:

Step S10: Determining in which operational setup of a plurality of predetermined operational setups the first medical device is operating.

Step S11: Determining a quality of service of a communication with the second device via the communication interface.

Step S12: Selecting one of the plurality of predetermined alarm modes based on the determined operational setup and the determined quality of service.

Step S13: Operating the first medical device in the selected alarm mode. Therein, in step S130 the controller of the first medical device determines whether an alarm message is present. If yes (Y), the method proceeds to step S131. If not (N), the method repeats step S130. In Step S131, the controller of the first medical device processes the alarm message in accordance with the selected alarm mode. For example, it produces an alarm, or suppresses it.

By the described solution the patient's comfort can be increased, and the caregiver's workload can be optimized while maintaining a high security level in compliance with alarm standard IEC/EN 606601 Jan. 8. Information exchange between the first medical device and the second device is secured. Unexpected network QoS issues can be detected.

The idea of the invention is not limited to the embodiments described above but may be implemented in a different fashion.

### LIST OF REFERENCE NUMERALS

1A-1E Medical device
10 Communication interface
11 Controller
12 Display
13 Sound emitting device
2 Caregiver device
20 Communication interface
21 Controller
22 Display
23 Sound emitting device
3 Mobile device
4 System
40 Hospital information system
41 Picture archiving and communication system
42 Clinical information system
43 Laboratory device
44 Communication device
5 Hospital network
6 Monitoring network
7 Server
A-E Operational setup
CR Caregiver room
OR Operating room
PR Patient room

1. A medical device comprising a communication interface communicatively couplable or coupled with another device, and a controller configured to be selectively operated in one of a plurality of predetermined alarm modes, wherein the controller is further configured to:
determine in which operational setup of a plurality of predetermined operational setups the medical device is operating;
determine a quality of service of a communication with the another device via the communication interface;
select one of the plurality of predetermined alarm modes based on the determined operational setup and the determined quality of service; and
operate the medical device in the selected one of the plurality of predetermined alarm modes.

2. The medical device according to claim 1, wherein the medical device is an infusion device or an infusion station for administering at least a fluid to a patient.

3. The medical device according to claim 1, wherein the controller is further configured to delegate or disseminate an alarm message to the another device depending on the determined operational setup and the determined quality of service.

4. The medical device according to claim 3, wherein the controller is further configured, in case of a delegated alarm message, to receive a response to the delegated alarm message from the another device.

5. The medical device according to claim 3, wherein the controller is further configured to produce a visible and/or audible alarm based on a delay of a response to the delegated alarm message and based on the selected one of the plurality of predetermined alarm modes.

6. The medical device according to claim 1, further comprising a display adapted to produce a visible alarm.

7. The medical device according to claim 1, further comprising a sound emitting device adapted to produce an audible alarm.

8. The medical device according to claim 1, wherein the plurality of predetermined alarm modes comprises a suppression mode, in which alarms are suppressed, and a perception mode, in which the alarms are perceivable.

9. The medical device according to claim 8, wherein the plurality of predetermined alarm modes comprises an attenuation mode, in which alarms are attenuated.

10. The medical device according to claim 1, wherein the controller is configured to determine in which operational setup of the plurality of predetermined operational setups the medical device is operating by at least one of:
receiving a user input at the medical device;
receiving a message indicating an operational setup via the communication interface; and
detecting a predetermined device or a predetermined plurality of devices via the communication interface.

11. The medical device according to any of the claim 1, wherein the controller is configured to determine the quality of service of a communication with the another device by at least one of:
receiving a response from the another device;
determining that a latency of a communication with the another device is above or below a predetermined latency threshold; and
determining that a network bandwidth of a communication with the another device is above or below a predetermined network bandwidth threshold.

12. The medical device according to claim 1, wherein the plurality of predetermined operational setups comprises at least one wireless setup, in which the communication interface communicates via a wireless connection with the another device, and at least one wired setup, in which the communication interface communicates via a wired connection with the another device.

13. A system, comprising:
at least one medical device in accordance with claim 1 and
another device communicatively coupled with the at least one medical device.

14. The system according to claim 13, further comprising a plurality of medical devices communicatively coupled with the another device via a same network.

15. The system according to claim 13, wherein the another device is a caregiver device adapted to visibly display and/or audibly emit alarms, and/or to send alarms to a mobile device.

16. A method for operating a medical device, the medical device comprising a communication interface communicatively couplable or coupled with another device, and a controller configured to be selectively operated in one of a plurality of predetermined alarm modes, the method comprising:
determining in which operational setup of a plurality of predetermined operational setups the medical device is operating;
determining a quality of service of a communication with the another device via the communication interface;
selecting one of the plurality of predetermined alarm modes based on the determined operational setup and the determined quality of service; and
operating the medical device in the selected one of the plurality of predetermined alarm modes.
